(19) United States
(12) Patent Application Publication
(10) Pub. No.: US 2026/0315743 A1
(43) Pub. Date: Oct. 15, 2026

(54) SUBCUTANEOUS TUNNELING OF A CATHETER

(71) Applicant: The Cleveland Clinic Foundation, Cleveland, OH (US)

(72) Inventors: Sean J. NAGEL, Cleveland Heights, OH (US); Mark LOBOSKY, Chardon, OH (US)

(21) Appl. No.: 19/476,303

(22) PCT Filed: Apr. 19, 2024
(86) PCT No.: PCT/US2024/025336

§ 371 Date: Oct. 17, 2025

Related U.S. Application Data
(60) Provisional application No. 63/460,430, filed on Apr. 19, 2023.

(51) Int. Cl. A61M 25/01; A61M 25/06; A61M 27/00

(52) U.S. Cl. CPC A61M 25/0194; A61M 25/0102; A61M 25/065; A61M 27/00; A61M 2202/0464; A61M 2210/1003

(57) ABSTRACT

An introducer needle assembly includes an inner shaft nested within an outer shaft. The inner shaft is repositionable relative to the outer shaft between a first orientation and a second orientation. In the first orientation, the inner and outer shafts define a window adjacent to a proximal end of the outer shaft that provides access to a common channel defined within the inner and outer shafts. In the second orientation, the inner and outer shafts define a slot that provides access to said common channel. The introducer needle is useful to facilitate a tunneling procedure wherein redirection of the tunneled catheter, for example, occurs entirely subcutaneously.

### CROSS-REFERENCE TO RELATED APPLICATIONS

This application claims the benefit of US provisional patent application No. 63/460,430 filed Apr. 19, 2023, which is incorporated herein by reference.

### FIELD OF THE INVENTION

This application relates generally to a needle assembly that can be used to introduce a surgical instrument (e.g., catheter) into a patient. More specifically, it relates to introducing said surgical instrument via a tunneling procedure, and subcutaneously redirecting said instrument via the needle assembly.

### BACKGROUND OF THE INVENTION

Catheters for introducing therapeutic agents or draining bodily fluids are ubiquitous in medicine. Conventionally, catheters can be inserted from a puncture wound at the patient's skin along a path leading directly to the point of therapy. Alternatively, catheters can be introduced via so-called tunneling procedures, so that the entrance puncture wound is located remote from the therapy site. The latter can be desirable to reduce the risk of infection at the therapy site, particularly when the catheter will be placed for an extended period.

A lumbar puncture (sometimes called a spinal tap) is a common procedure that involves introducing a catheter into the dura of a patient's spinal column. It can be used to either (or both) administer medication or to drain cerebrospinal fluid. To insert the catheter, one first inserts a needle (e.g., a Touhy needle) through the skin (targeting the intrathecal space) and punctures the spinal dura mater to introduce a distal end of a catheter therein through the needle. Thereafter, the needle is removed and the puncture wound at the skin through which the catheter passes may be sewn closed around the catheter (or otherwise protected e.g., via bandages, tape, etc.). In a straight-line procedure, the puncture in the spinal dura mater and the skin-puncture wound are located near one another along a linear path followed by the needle and thereafter the catheter. However, when the catheter will be emplaced for prolonged periods of time (e.g., greater than 48 hours), such proximity can be undesirable due to the risk of infection migrating from the skin-puncture wound to the nearby dura puncture along a short path. In addition, there is an increased risk that the cerebrospinal fluid will track along the catheter. In such instances, therefore, a tunneling procedure can be preferred to locate the skin-puncture wound remote from where the dura is punctured.

In a conventional tunneling procedure, the catheter is inserted into the dura mater (via an introducer needle, e.g. a Tuohy needle) from a first puncture wound in the vicinity of where it enters the dura, and then back-threaded through a tunneller (or a second Tuohy needle) that is subcutaneously ‘tunneled’ laterally from a second puncture wound located remote from the first. The tunneller is directed subcutaneously toward the first puncture wound in order that the catheter can be threaded therein, ultimately to exit the patient's body via the second, remote, skin-puncture wound. FIGS. 15-17 show a conventional tunneller 200 used for this purpose. The tunneller 200 includes a metallic probe 202 connected to a handle 204, and a removable sheath 206 that circumferentially surrounds and accommodates the probe 202 when assembled. During an operation, the tunneller 200 (i.e. its probe 202 and the surrounding sheath 206) is inserted via the second skin-puncture wound noted above, at a location remote from where the first skin-puncture wound is to be made in the vicinity of where the spinal dura mater is to be punctured. The tunneller is advanced subcutaneously along a declined lateral path until its tip intersects an imaginary line that is substantially perpendicular to the probe and intersects the dura-puncture target. Thereafter, an incision is made in the skin to yield the aforementioned first skin-puncture wound at a location along that imaginary line, in the vicinity of the dura-puncture target, to expose the tip of the probe 202 under the skin.

Next, the probe 202 is withdrawn leaving in-place the sheath 206, which establishes a lateral path between the remote, second skin-puncture wound a region beneath the first puncture wound. A needle (such as a Touhy needle) then can be inserted via the first puncture wound toward the dura-puncture target to puncture the spinal dura mater. In conjunction therewith, the catheter is introduced into the proximal end of the needle and threaded down until its distal end arrives within the dura mater via the associated puncture wound and then advanced therein (e.g., under fluoroscopic guidance). Once the catheter is in-place, the introducer (e.g. Touhy) needle is removed from the patient. Next, a proximal (i.e., free) end of the catheter is fed into a distal end of the sheath 206 located in the vicinity of the first skin-puncture wound and threaded retrograde therethrough until it emerges from a proximal opening 208 of the sheath 206 in the vicinity of the remote, second skin-puncture wound. The sheath 206 is then removed from the patient, resulting in the proximal end of the catheter being exposed at the remote skin-puncture wound. Finally, both skin wounds are closed.

This procedure results in the puncture in the spinal dura mater and the insertion point of the catheter through the skin being remote from one another, thereby minimizing the potential for an infection where the catheter penetrates the skin to migrate to the dura puncture. However, this procedure still requires introducing the proximal end of the catheter through the first puncture wound and threading it beneath the skin to the remote puncture-wound site.

### BRIEF SUMMARY

In accordance with one aspect, there is provided an introducer needle assembly including a hollow outer shaft and a hollow inner shaft nested within said outer shaft. The inner shaft is repositionable relative to the outer shaft between a first orientation and a second orientation. In the first orientation, the inner and outer shafts define a window adjacent to a proximal end of the outer shaft that provides access to a common channel defined within the inner and outer shafts. In the second orientation, the inner and outer shafts define a slot that provides access to the common channel, wherein the slot extends from adjacent to the proximal end to a distal end of the outer shaft.

In accordance with another aspect, there is provided a method of introducing a catheter into a desired location of a patient. The method includes inserting an introducer needle assembly into the patient at a first skin location and along a first trajectory targeting a predetermined target tissue region within a body of the patient, the introducer needle assembly being in a first configuration wherein a window is defined adjacent to a proximal end thereof that provides access to a channel therein. The method further includes inserting a tunneller at a second skin location, remote from the first skin location, and advancing the tunneller subcutaneously along a second trajectory that is angled with respect to the first trajectory and targets the window. Further, the method includes advancing a catheter through the tunneller along the second trajectory and through the window. Further, the catheter is redirected from the second trajectory along the first trajectory within the channel, and advanced from its point of entry at the second skin location, along the second and first trajectories successively, until a distal end of the catheter reaches the target tissue region.

In accordance with a further aspect, there is provided an introducer needle assembly including a hollow outer shaft having an outer cutout extending longitudinally in a circumferential wall thereof from adjacent to a proximal end thereof to a distal end thereof. A first head is affixed to the proximal end of the outer shaft. The first head has a bore, wherein a boss is formed on an inner circumferential wall of the bore and projects radially inward. The assembly further includes a hollow inner shaft nested within the outer shaft. The inner shaft includes a circumferential wall having a slotted portion and a window portion. The slotted portion includes an inner cutout extending longitudinally in the circumferential wall of the inner shaft such that the circumferential wall is discontinuous and has a first arc length in the slotted portion. The circumferential wall is confined to a narrow wall segment having a second circumferential arc length in the window portion, wherein the second circumferential arc length is less than the first circumferential arc length. A second head is affixed to a proximal end of the inner shaft. The second head has a neck that is received within the bore. The neck has a radially recessed detent extending along a circumferential arc segment thereof and defined between opposing first and second stops, wherein the boss is received within the detent. The inner shaft is rotatable relative to the outer shaft between a first orientation and a second orientation. In the first orientation, the inner and outer shafts define a window adjacent to the proximal end of the outer shaft that provides access to a common channel defined within the inner and outer shafts. Further, the inner and outer cutouts of the inner and outer shafts, respectively, are not aligned, and the window is defined in-part by an edge of the circumferential wall in the slotted portion of the inner shaft that traverses the outer cutout in the outer shaft. Further still, the boss engages the first stop. In the second orientation, the inner and outer shafts define a slot that provides access to the common channel, wherein the slot extends from adjacent to the proximal end of the outer shaft to the distal end of said outer shaft. Further, the inner and outer cutouts of the inner and outer shafts, respectively, are aligned to thereby define the slot, and the boss engages the second stop.

### BRIEF DESCRIPTION OF THE DRAWINGS

FIG. 1 is a perspective view of a catheter introducer needle assembly as herein disclosed;

FIG. 2 is a further perspective view of the needle assembly shown in FIG. 1, which is partially in section along the line 2-2 in FIG. 1;

FIG. 3 is an exploded view of the needle assembly of FIG. 1, including outer and inner needle portions and a stylet;

FIG. 4A is a perspective view of the outer needle portion shown in FIG. 3;

FIG. 4B is another perspective view of the outer needle portion shown in FIG. 3;

FIG. 4C is an end view showing a proximal end of the outer needle portion shown in FIG. 3;

FIG. 5A is a perspective view of the inner needle portion shown in FIG. 3;

FIG. 5B is another perspective view of the inner needle portion shown in FIG. 3;

FIG. 5C is an end view showing a distal end of the inner needle portion;

FIG. 5D is an end view showing a distal end of an alternative inner needle portion;

FIG. 6 is a perspective view of the stylet shown in FIG. 3;

FIG. 7 is a side view of the needle assembly, wherein the needle assembly is in a first (closed) configuration defining a window;

FIG. 8 is another side view of the needle assembly, wherein the needle assembly is in a second (opened) configuration defining a slot that extends from adjacent to a proximal end to a distal end;

FIG. 9 is a schematic depiction of the needle assembly inserted within a patient;

FIG. 10 is another schematic view of the needle assembly inserted in the patient;

FIG. 11 is a schematic view showing a catheter being inserted and directed subcutaneously toward the window of the needle assembly from a remote location (which can be via a tunneller);

FIG. 12 is a schematic view as in FIG. 11, but depicting the catheter having been advanced through the needle assembly so that its distal end enters the spinal dura mater,

FIG. 13 is a schematic view as in FIG. 12, wherein the needle assembly has been actuated to its second (opened) orientation, and is being withdrawn from the patient leaving behind the catheter;

FIG. 14 is a schematic depiction as in FIG. 13, wherein the catheter remains in-place following complete withdrawal of the needle assembly;

FIG. 15 is a side view of a tunneller;

FIG. 16 is an exploded view of the tunneller shown in FIG. 15;

FIG. 17 is a perspective view of a sheath; and

FIG. 18 is a perspective view of an alternative embodiment of a stylet.

### DESCRIPTION OF EXAMPLE EMBODIMENTS

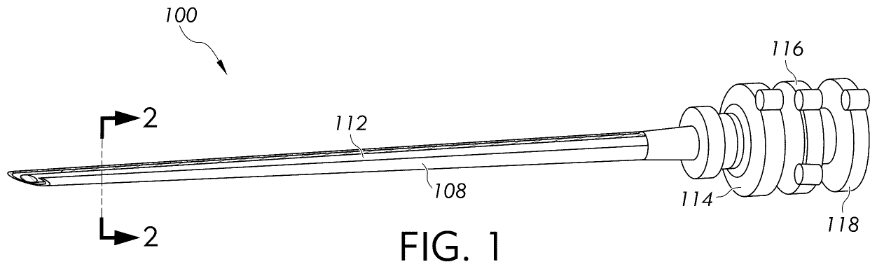

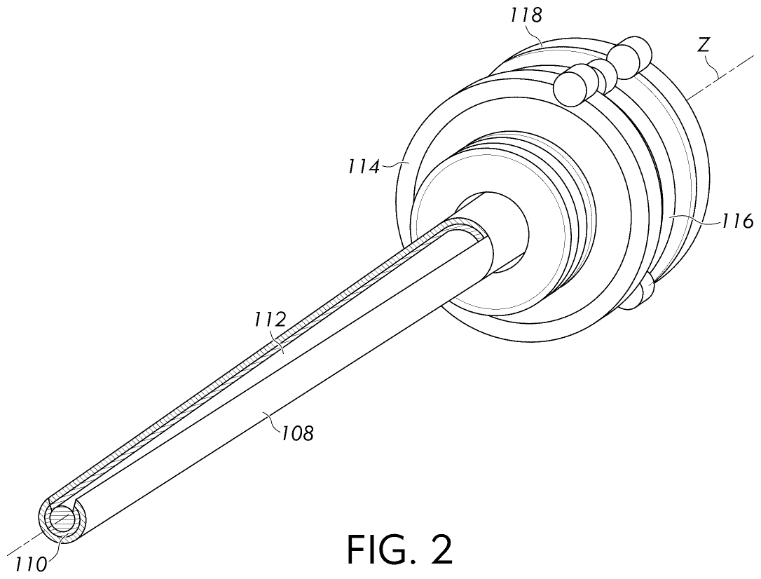

With reference to FIGS. 1-3, illustrated is catheter introducer needle assembly 100, which can replace a conventional Touhy needle for redirecting a tunneled catheter toward (and optionally into) a target body space for appropriate therapy. In the illustrated embodiment, the needle assembly 100 is a nested assembly 100 having an outer needle portion 102, an inner needle portion 104, and a stylet 106. Each of the outer and inner needle portions 102, 104 and the stylet 106 has a respective shaft 108, 110, 112 and a respective head 114, 116, 118 provided at a proximal end thereof. As shown in FIG. 2, depicting a partial cross-sectional view of the needle assembly 100, the outer and inner needle portions 102, 104 and the stylet 106 are coaxial and concentrically nested along a common ‘Z’ axis. More specifically, the shaft 112 of the stylet 106 is received within the shaft 110 of the inner needle portion 104, which itself is received within the shaft 108 of the outer needle portion 102 when assembled together. As further explained below, each of the inner needle portion 104 and the stylet 106 is movable (i.e., translatable along and rotatable relative to the ‘Z’axis), independently of one another and of the outer needle portion 102.

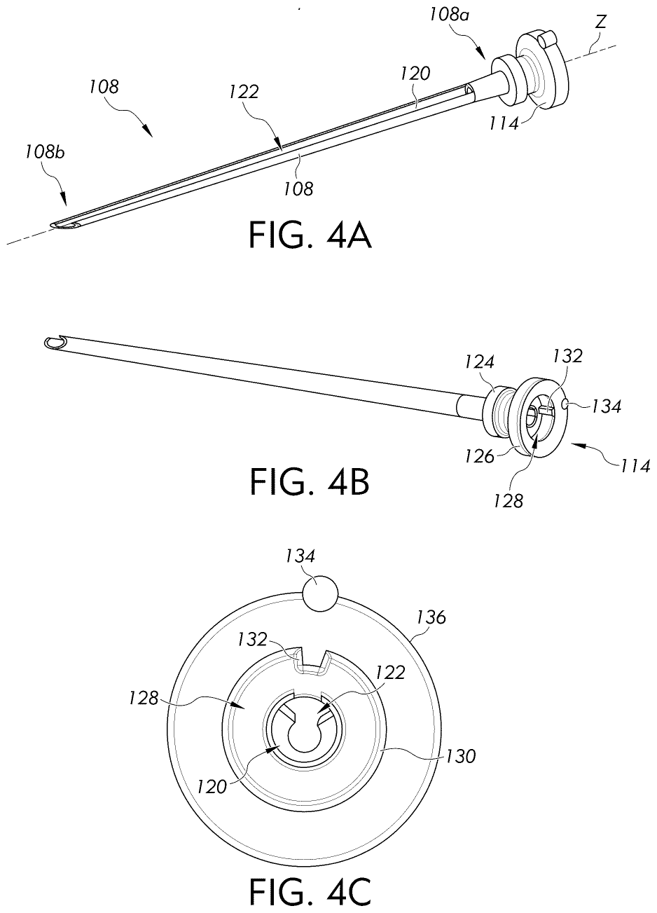

Referring now to FIGS. 4A-4C (depicting various views of the outer needle portion 102), the shaft 108 extends from a proximal end 108a to a distal end 108b. The head 114 is disposed at the proximal end 108a, and the distal end 108b is formed to have a pointed tip. The shaft 108 is essentially a hollow tube, having a circumferential wall that defines an outer channel 120 therein. The outer channel 120 extends completely through the shaft 108, and is configured to receive the respective shafts 110, 112 of the inner needle portion 104 and the stylet 106 nested therein.

In one embodiment, the shaft 108 may be cylindrical such that its circumferential wall has a constant radius of curvature between its proximal and distal ends 108a, 108b. Alternatively, as depicted in FIGS. 4A-4B, the shaft 108 may be cone-shaped, such that its circumferential wall has a radius of curvature that decreases toward the distal end 108b. Notably, that radius of curvature can decrease continuously from the proximal end 108a to the distal end 108b, or it can decrease along only a portion of the length there between; e.g., within a convergent region of the shaft 108, wherein other region(s) of the shaft 108 has/have a constant radius of curvature.

As further shown in FIGS. 4A-4C, a longitudinally oriented cutout 122 (referred to below as an outer cutout) extends longitudinally in the circumferential wall of the shaft 108, from a location preferably adjacent to its proximal end 108a all the way to the distal end 108b.

As shown best in FIGS. 4B and 4C, the head 114 includes a neck 124 affixed to the proximal end 108a of the shaft 108, and a collar 126 projecting radially outward. A bore 128 in the collar 126 provides access to the channel 120 defined by the shaft 108 of the outer needle portion 102, at the proximal end 108a thereof. That bore 128 also is configured to accommodate a neck of the head 116 of the inner needle portion 104, as discussed further below.

A boss 132 is formed on an inner circumferential wall 130 of the bore 128 and projects radially inward thereof. Moreover, a boss indicator 134 can be provided on an outer circumferential wall 136 of the collar 126. In the illustrated embodiment, the boss indicator 134 is circumferentially aligned with the boss 132, and is formed as a protrusion that projects radially outward from the outer circumferential wall 136. But the boss indicator 134 may simply be a marking on that wall 136. Each of the boss 132 and the boss indicator 134 may be formed integral with the collar 126 (i.e., as a single piece part).

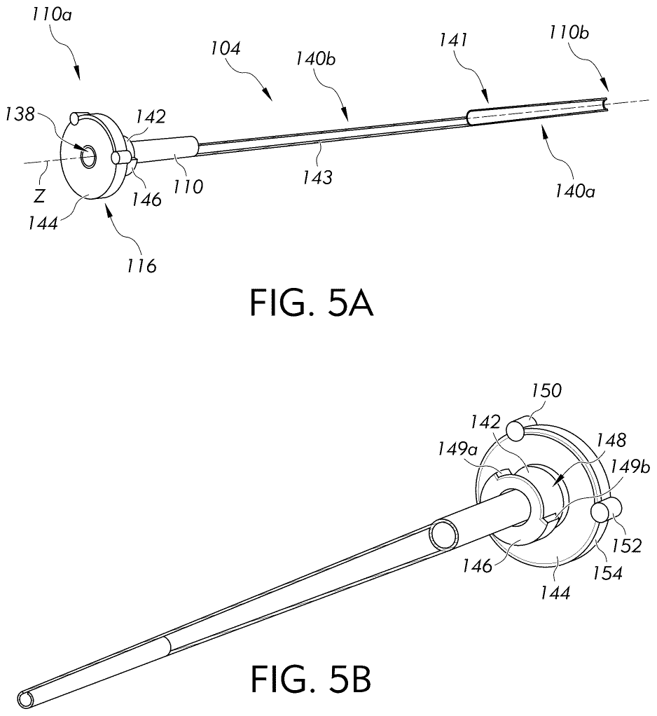

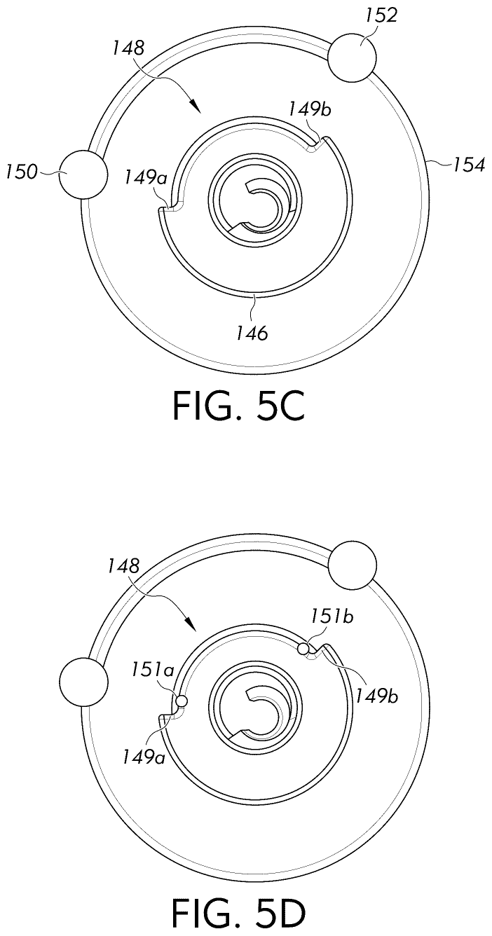

Now referring to FIGS. 5A-5C (depicting various views of the inner needle portion 104), the shaft 110 of the inner needle portion 104 extends from a proximal end 110a to a distal end 110b, wherein the head 116 is disposed at the proximal end 110a. Similar to the shaft 108 of the outer needle portion 102, the shaft 110 of the inner needle portion 104 has a circumferential wall that defines a channel 138 configured to receive the shaft 112 of the stylet 106. The shaft 110 of the inner needle portion 104 may be cylindrical or cone-shaped (as shown in FIGS. 5A and 5B), such that its shape will be complementary to that of the shaft 108 of the outer needle portion 102. In this manner, when the outer and inner needle portions 102, 104 are nested, their respective shafts 108, 110 will be arranged complementary so that the inner shaft 110 is fitted against the inner wall of the shaft 108 of the outer needle portion 102.

The shaft 110 of the inner needle portion 104 is generally tubular (again, preserving complementarity with the shaft 108 of the outer needle portion 102), and defines a slotted portion 140a adjacent to its distal end 110b, and a window portion 140b remote from the distal end 110b, and preferably adjacent to the proximal end 110a. In the slotted portion 140a, an inner cutout 141 is disposed in the circumferential wall of the shaft 110 and extends longitudinally along the ‘Z’ axis. Accordingly, the circumferential wall of the shaft 110 is discontinuous in the slotted region as seen in FIG. 5A. The inner cutout 141 preferably has a comparable width (in radians) compared to the outer cutout 122 in the shaft 108 of the outer needle portion 102, although it may be slightly wider. Whereas, in the window portion 140b, the circumferential wall of the shaft 110 preferably is cut away except for a narrow segment 143 of that wall that extends continuously through the window portion 140b, from adjacent to the proximal end 110a to the slotted portion 140a of the shaft 110. The narrow wall segment 143 extending through the window portion 140b has a circumferential arc length preferably not more than π/2 radians, more preferably not more than π/4 radians or π/8 radians. Alternatively, in the window portion 140b a slot of substantially greater width (i.e., in radians) may be provided in communication with the inner cutout 141 in the slotted portion 140a without being so substantial as to reduce the circumferential wall to merely the narrow segment 143 illustrated in FIG. 5A. In any event, the arc length of the portion of the circumferential wall in the window portion 140b is smaller than the arc length of that wall in the slotted portion 140a.

As further shown, the head 116 of the inner needle portion 104 is affixed to the shaft 110 at its proximal end 110a. The head 116 includes a neck 142 extending distally from a distal face of a collar 144. The collar 144 projects radially outward from the neck 142. In the illustrated embodiment, the neck 142 is generally cylindrical in shape having a first radius along a first circumferential arc segment 146 of the neck 142, and a radially recessed detent portion 148 having a second radius, smaller than the first radius, that extends along a second circumferential arc segment of the neck 142. Respective detent stops 149a and 149b are defined at the opposing interfaces between the portions of the neck 142 having the respective first and second radii. See FIGS. 5B and 5C.

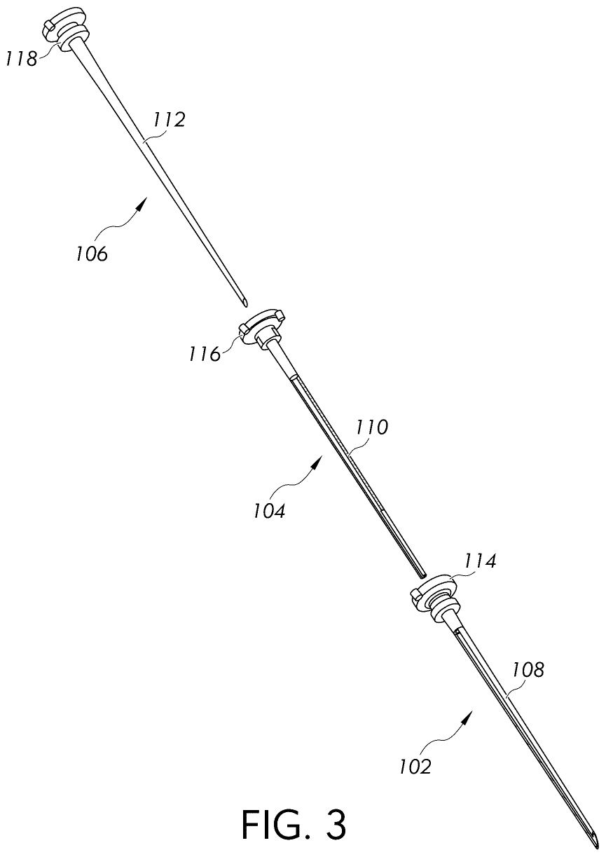

As will be appreciated with reference to at least FIGS. 3, 4A and 5B, when the outer and inner needle portions 102 and 104 are nested such that the shaft 110 of the inner needle portion 104 is received and accommodated within the shaft 108 of the outer needle portion 102, the neck 142 of the inner needle portion 104 is received and rotationally accommodated within the bore 128 of the collar 126 of the outer needle portion 102. The neck 142 is seated within the bore 128 such that the boss 132 is disposed within the detent portion 148 of neck 142, confined between the opposing stops 149a and 149b. In this manner, rotation of the inner needle portion 104 (and its collar 126) is permitted only to the extent that the boss 132 can navigate an arcuate path between the opposing stops 149a and 149b, beyond which it cannot proceed further. Preferably, status indicators 150, 152 are provided at an outer circumferential wall 154 of the collar 144, corresponding to, and preferably circumferentially aligned with, the relative circumferential positions of the respective stops 149a, 149b.

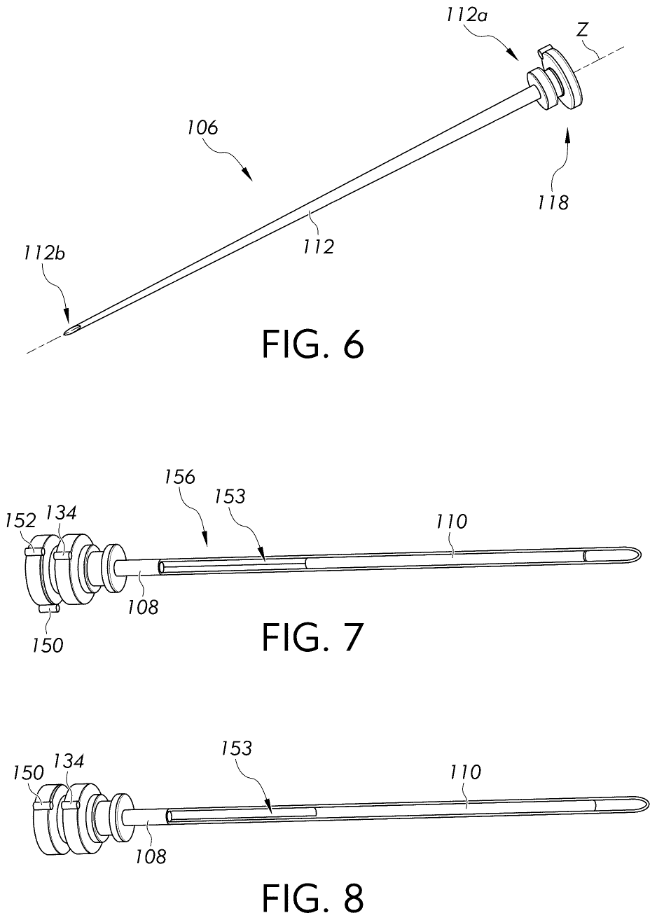

Now with reference to FIG. 6, the shaft 112 of the stylet 106 extends from a proximal end 112a to a distal end 112b, wherein the head 118 is disposed at the proximal end 112a and the distal end 112b can be formed to have a pointed tip complementary to the tip defined at the distal end 108b of the shaft 108 of the outer needle portion 102. That is, when fully nested the tip portions of each of the shaft 108 (of the outer needle portion 102) and the shaft 112 (of the stylet 106) together can define a common-tip for puncturing flesh, such as the skin and/or the dura mater for introduction of a catheter. Alternatively, the complete assembly (including outer needle portion 102, inner needle portion 104 and stylet 106) can be configured to have a blunt tip when assembled, as opposed to a pointed one. This may be preferred, for example, in cases when the clinician intends to use the assembly within the epidural space in order to reduce risk of dural puncture while inserting a tunneled epidural wire or catheter, for example.

Like the shaft 108 of the outer needle portion 102, the shaft 112 of the stylet 106 may be cylindrically shaped, or alternatively it may be generally cone-shaped as discussed above, in order to ensure complementary nesting of the components. However, unlike the respective shafts 108, 110 of the outer and inner needle portions 102, 104, the shaft 112 of the stylet 106 is a non-hollow member and imparts rigidity to the needle assembly 100 to facilitate penetration into flesh to direct its distal end to the desired target location; e.g., where it can puncture the dura mater to facilitate catheter placement.

As mentioned above, when the shaft 110 of the inner needle portion 104 is received within the channel 120 of the outer needle portion 102, the neck 142 of the head 116 (shown in FIG. 5B) is received within the bore 128 in the head 114 (shown in FIGS. 4B-4C) such that the boss 132 is disposed within the detent portion 148 between the opposing stops 149a and 149b. During operation, the needle assembly 100 is actuatable or adjustable (i.e., via rotation) between a first (closed) configuration and a second (opened) configuration. More specifically, the shaft 110 of the inner needle portion 104 is repositionable relative to the shaft 108 of the outer needle portion 102 between a first orientation (corresponding to the first configuration of the overall assembly 100) and a second orientation (corresponding to the second configuration of the assembly 100). Notably, rotation of the head 116 of the inner needle portion 104 relative to the head 114 of the outer needle portion 102 is confined based on the available arcuate travel path of the boss 132, between the opposing stops 149a and 149b that bound the detent portion 148 of the head 116 (of the neck 142).

In the illustrated embodiment, when the outer and inner needle portions 102 and 104 are nested, and the boss 132 abuts the stop 149b on the neck 142, the needle assembly 100 is provided in the first configuration (as shown in FIG. 7, which omits the stylet 106). In the first configuration, the window portion 140b of the shaft 110 of the inner needle portion 104 and the outer cutout 122 formed in the outer needle portion 102 are aligned such that the shaft 110 does not obstruct a window 156 (see FIG. 7) through the shafts 108, 110 leading to a common channel 153 defined at the center of both those shafts 108, 110. Notably, the window 156 does not longitudinally extend (i.e., along the ‘Z’ axis) the entire extent of the needle assembly 100. Rather, in the first configuration, the inner cutout 141 in slotted portion 140a of the inner needle portion 104 does not circumferentially align (i.e., overlap) with the outer cutout 122 formed in the outer needle portion 102. As a result, as seen in FIG. 7, a distal segment of the needle assembly 100, between the window 156 and the tip of the shaft 108 of the outer needle portion 102, remains closed off so that penetration through the outer cutout 122 in the shaft 108 of the outer needle portion 102 is inhibited by the shaft 110 of the inner needle portion 104 obstructing that outer cutout 122 in the distal segment. Succinctly, a proximal edge of the circumferential wall in the slotted portion 140a of the inner shaft 110 partially defines the window 156 by traversing the outer cutout 122 in the outer shaft 108. The overall result is that in the first configuration, the needle assembly 100 presents a needle having an access window 156 at a proximal location along the needle shaft (defined between the shafts 108 and 110), but which is otherwise a closed cylindrical needle leading to its distal tip.

By rotating the inner needle portion 104 (about the ‘Z’ axis) with respect to the outer needle portion 102, such that the boss 132 follows an arcuate path within the detent portion 148 until it abuts the stop 149a on the neck 142, the needle assembly 100 is provided in the second (opened) configuration as shown in FIG. 8. In the second configuration, the full length of the outer cutout 122 provides access to the common channel 153 defined at the center of both the shafts 108, 110 of the respective outer and inner needle portions 102 and 104, from adjacent to the proximal ends thereof all the way to and through the distal tip. More specifically, in the second configuration, the inner and outer cutouts 141, 122 of the inner and outer shafts 110, 108, respectively, are aligned to thereby define a slot that provides access to the common channel 153.

The boss indicator 134 of the outer needle portion 102 is aligned relative to the boss 132 (e.g., it can be circumferentially aligned as shown best in FIG. 4C, though this is not required), so that its circumferential location corresponds to or indicates the circumferential location of the boss 132. Separately, the status indicators 150, 152 of the inner needle portion 104 are circumferentially aligned relative to (optionally they can be circumferentially aligned with) the stops 149a, 149b that bound the detent portion 148 of the neck 142, respectively (shown best in FIG. 5C). Accordingly, when the needle assembly 100 is placed in the closed configuration (with the boss 132 abutting the stop 149b), the boss indicator 134 will align with the status indicator 152 of the inner needle portion 104 (as shown in FIG. 7). Alternatively, when the needle assembly 100 is placed in the opened configuration (with the boss 132 abutting the stop 149a), the boss indicator 134 will align with the status indicator 150 of the inner needle portion 104 (as shown in FIG. 8). Accordingly, alignment of the boss indicator 134 with either of the status indicators 150, 152 of the inner needle portion 104 provides an indication to the user as to whether the needle assembly 100 is in the closed configuration or the opened configuration. To enhance such an indication, the design of the status indicators 150, 152 may differ from one another. For example, the status indicators 150, 152 may have different colors, shapes, textures, etc. For example, in the illustrated embodiment, the red status indicator 152 corresponds to the closed configuration, whereas the green status indicator 150 corresponds to the opened configuration.

As noted above, the inner needle portion 104 is freely rotatable with respect to the outer needle portion 102, wherein such movement is bounded by the respective engagements between the stops 149a, 149b and the boss 132. With reference to FIG. 5D, the detent portion 148 of the neck 142 may include nubs 151a, 151b disposed adjacent to the stops 149a, 149b, respectively. The nubs 151a, 151b protrude slightly from the radiused wall of the neck 142 in the detent portion 148 thereof, and are spaced from the respectively adjacent stop 149a, 149b by a small gap. The gaps are configured to receive the boss 132 therein, bounded between the adjacent stop 149a, 149b and its associated nub 151a, 151b. Each of the nubs 151a, 151b may be dimensioned so as to provide mild resistance when engaging with the boss 132 during rotation of the inner needle portion 104. That is, as the boss 132 reaches one of the nubs 151a, 151b, the user will need to apply additional force to place the needle assembly 100 in the respective closed or opened configuration. After the boss 132 is received in the corresponding gap, the needle assembly 100 will be reversibly locked in the corresponding configuration, thereby minimizing potential accidental rotation of the inner needle portion 104.

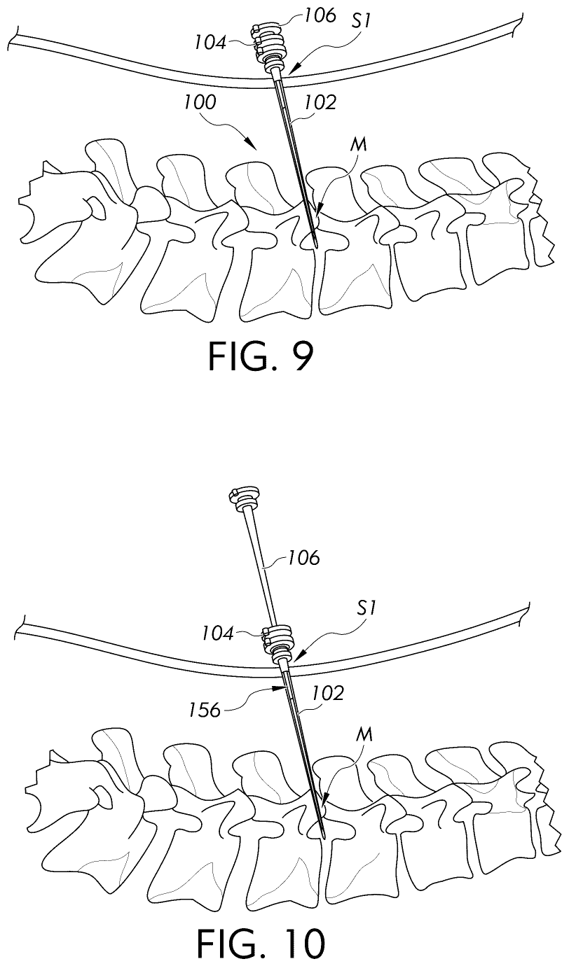

A method of using the needle assembly 100 to introduce a catheter into the desired location via a tunneling procedure will now be described with reference to FIGS. 9-14. Notably, FIGS. 9-14 are schematic representations of the needle assembly 100 and a tunneller 200 as discussed above. First, the needle assembly 100 is inserted into a patient at a first skin location ‘S1’ adjacent to where the distal end of the catheter is to be emplaced. Preferably the needle assembly 100 is inserted along a first trajectory targeting a predetermined target tissue region within the patient. Here, the needle assembly 100 is inserted as shown, directly targeting the location where the distal end of the catheter is to be introduced; e.g., the intrathecal space within the spinal dura. That is, the needle assembly 100 is inserted along the first trajectory and punctures both the skin (at the first skin location ‘S1’) and the spinal dura mater (at a location ‘M’) thereby resulting in substantial axial alignment of the insertion site at the first skin location S1 and the puncture ‘M’ in the spinal dura mater. Accordingly, the first trajectory is linear or substantially linear. Notably, during insertion, the needle assembly 100 is placed in the first (closed) configuration (i.e., shown in FIG. 7), and the stylet 106 is received within the inner needle portion 104. As mentioned above, the outer and inner needle portions 102, 104 together define a common (hollow) channel 153, whereas the stylet 106 is solid. By nesting the solid stylet 106 within the nested outer and inner needle portions 102, 104, the needle assembly 100 is rigidified, thereby enhancing ease of insertion and minimizing the risk of buckling either of the outer or inner needle portions 102, 104. The stylet 106 also minimizes the potential of coring the patient's flesh upon insertion.

Insertion of the needle assembly 100 can be aided via fluoroscopic guidance. With respect to FIG. 10, after insertion, the stylet 106 is backed-out or removed. Notably, the stylet 106 may be backed-out only to a certain degree (e.g., as indicated by a mark on the shaft of the stylet 106, not shown) or it may be removed completely. Removing or backing-out the stylet 106 serves two purposes. First, by doing so, the operator (e.g., surgeon, etc.) can confirm placement within the intrathecal space (as cerebrospinal fluid will flow out of the needle assembly 100). After confirmation, the stylet 106 may be reintroduced into the inner needle portion 104 to block the flow of cerebrospinal fluid. Withdrawing the stylet 106 just beyond the window 156, on the other hand, provides access to the common channel 153 within the needle assembly 100 via that window 156.

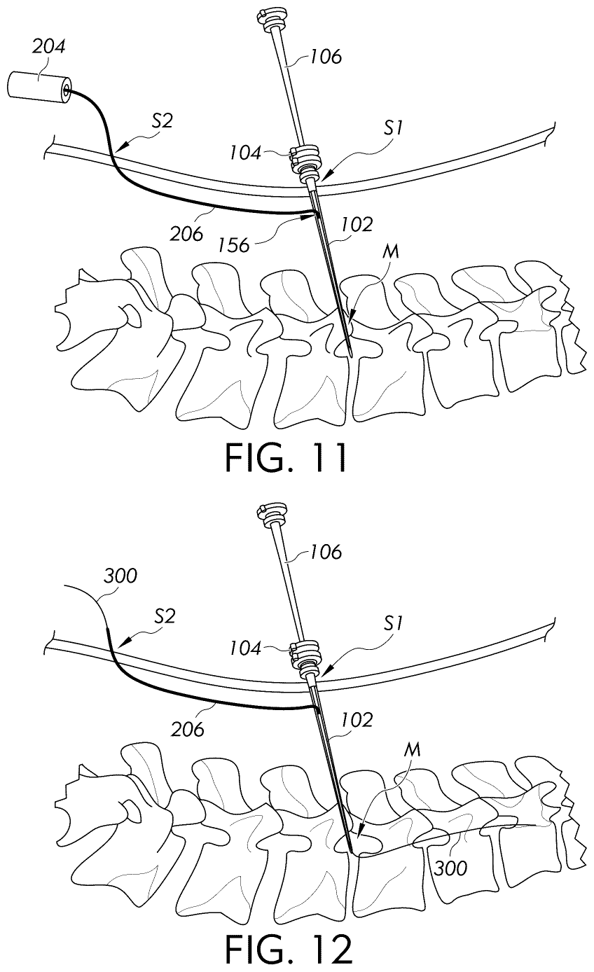

Subsequently, with respect to FIG. 11, a conventional tunneller (as described above) is inserted at a second skin location ‘S2’ remote from the first skin location ‘S1,’ and advanced laterally along a second trajectory at an (preferably downward) angle (relative to the first trajectory) to target the window 156 of the needle assembly 100. Guidance of the probe 202 of tunneller 200 can be achieved via known techniques, e.g., fluoroscopic and/or tactile feedback, until it reaches the common channel 153 of the needle assembly 100 via the window 156. Notably, the sheath 206 (and probe 202) of the tunneller is (are) received within the common channel 153 forming an obtuse angle with the needle assembly 100. This orientation facilitates threading the distal end of the catheter into and down the common channel 153, as discussed below. As appreciated, the second skin location ‘S2’ is laterally offset from the puncture ‘M’ in the spinal dura mater and completely remote from the first skin location ‘S1’. The sheath 206 can be rigid or flexible, though rigid may be preferred because it will be easier to manipulate and less likely to stray off course once positioned subcutaneously and the once the probe 202 is removed; see next paragraph. A rigid sheath 206 also will be easier to advance subcutaneously on its own as desired once that probe has been removed.

Thereafter, with reference to FIG. 12, the tunneller probe 202 is removed, leaving in place the sheath 206, which provides a pathway from the puncture wound at the second skin location ‘S2’ into the common channel 153 of the needle assembly 100 via the window 156 (i.e., along the second trajectory). Next, the catheter 300 is snaked through the sheath 206 (via the proximal opening 208), and into the common channel 153 of the needle assembly 100 via the window 156. The catheter 300 can be further fed so that it is redirected from the second trajectory, downward along the first trajectory and through the common channel 153 of the needle assembly 100, until its distal end is received in the intrathecal space. The obtuse angle between the sheath 206 and the needle assembly 100 helps the catheter 300 to negotiate the directional change from the former into the latter.

The catheter 300 may be guided downward through the common channel 153 of the needle assembly 100 purely via advancement from the proximal end thereof, which emerges from the skin at the second skin location ‘S2’. Alternatively, after the distal end of the catheter 300 is received in the common channel 153, the tipped-stylet 106 (shown in FIG. 6) may be removed and replaced by a blunt stylet (shown in FIG. 18) that may be used to further facilitate the catheter's guidance via plunging.

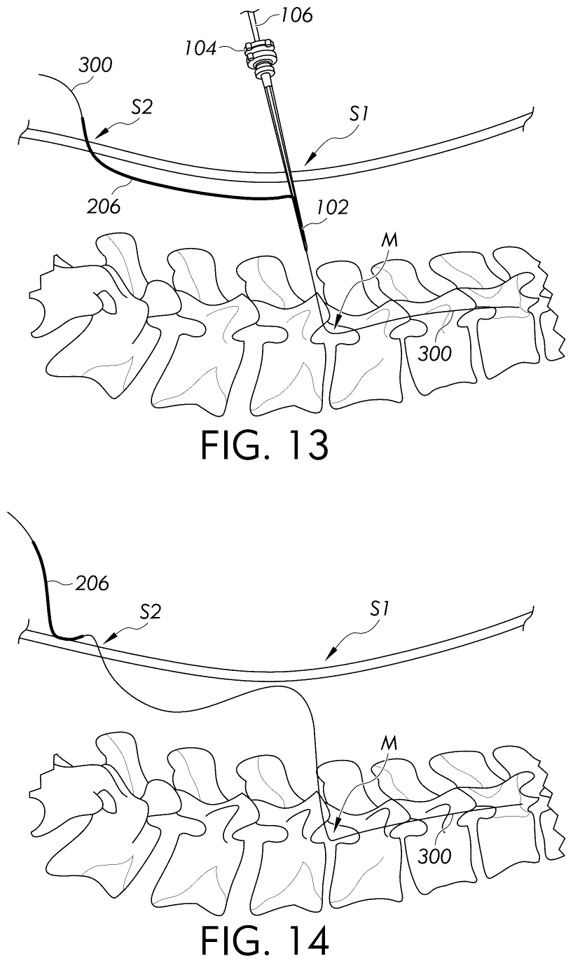

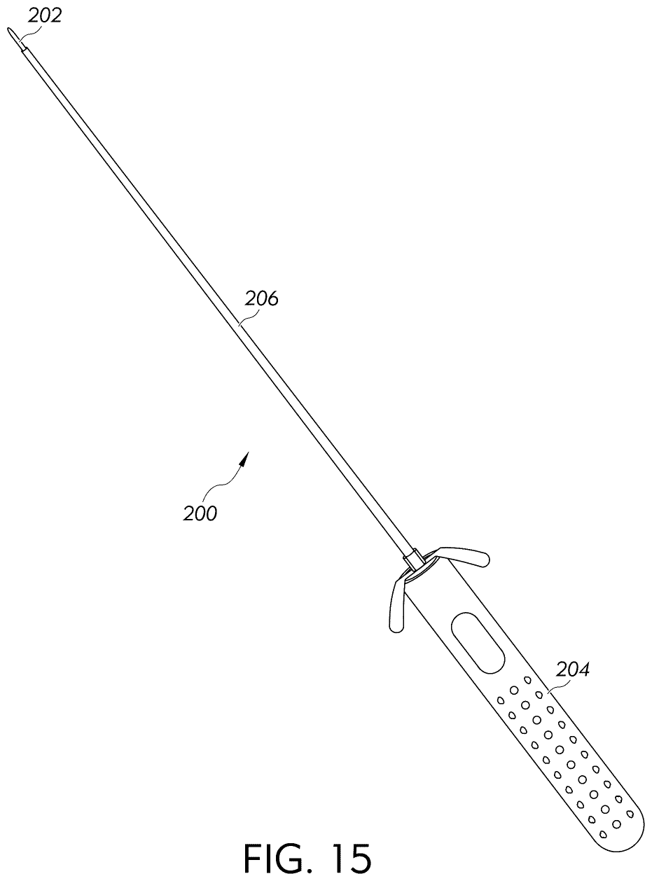

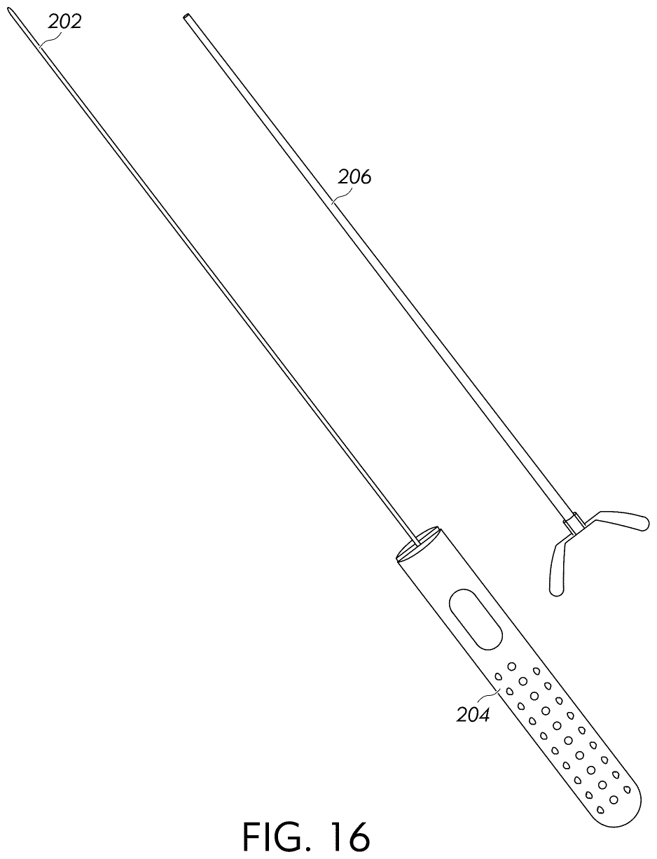

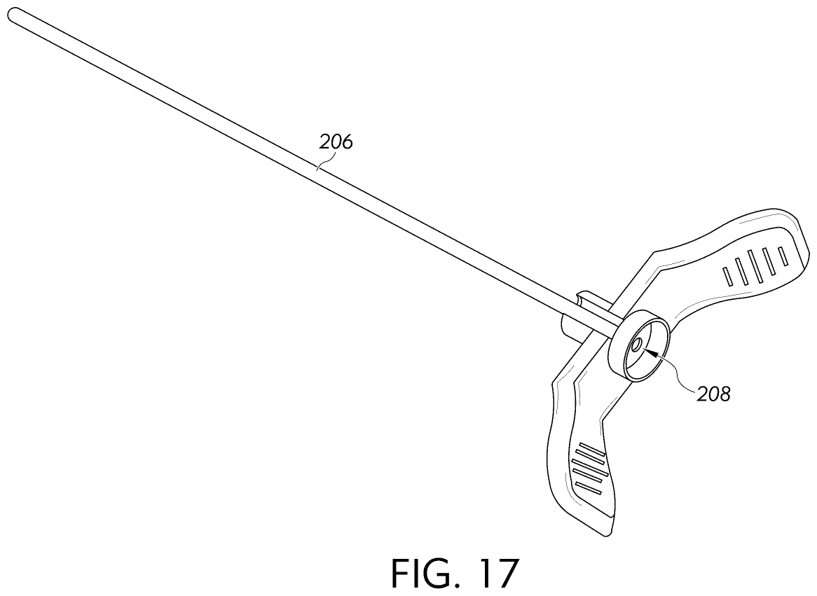

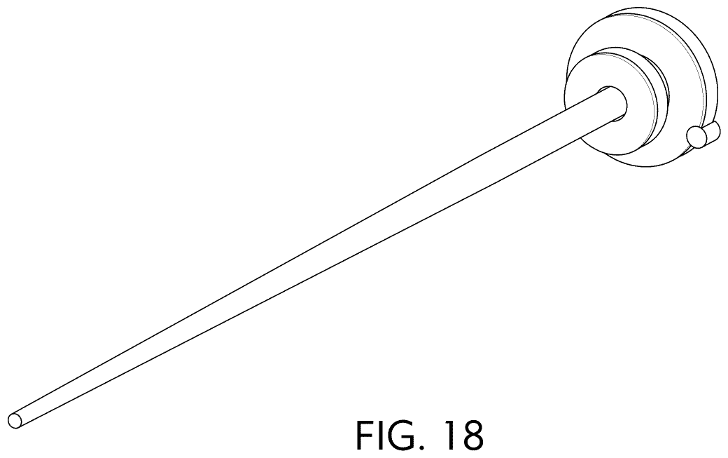

Subsequently, with respect to FIG. 13, the needle assembly 100 is actuated from the first (closed) configuration (as shown in FIG. 7) to the second (opened) configuration (as depicted in FIG. 8) by rotating the inner needle portion 104 with respect to the outer needle portion 102, as discussed above. Doing so completely opens the full length of the outer cutout 122 (thus defining the slot in the assembly 100), providing access to the common channel 153 defined at the center of both the shafts 108, 110 along substantially its entire length from where the window 156 was located in the first configuration.

At this point, the needle assembly 100 is removed from the patient along the linear pathway by which it was inserted (i.e., the first trajectory), whereupon the emplaced catheter 300 passes freely through the fully exposed outer cutout 122 permitting removal of the needle assembly 100 without disturbing the catheter 300. The skin wound at the first skin location ‘S1’ can be closed (e.g., sutured), as depicted in FIG. 14. Finally, the sheath 206 of the tunneller is withdrawn over the catheter 300, and out of the skin wound at the second skin location ‘S2,’ leaving the catheter 300 in place. That skin wound also can be closed (e.g., sutured) in a conventional manner around the catheter 300 emerging from that wound.

Notably, prior to withdrawing the needle assembly 100 from the patient, it can be used to insert a second or further catheter into the patient at the same targeted site (i.e., spinal dura) without removing the needle assembly 100 from the patient. Specifically, after the first catheter has been placed as described above, the needle assembly 100 can be adjusted to its second configuration, and the first current catheter can be urged out of the common channel 153 therein via a stylet 106 (e.g. shown in FIG. 6 or FIG. 18) inserted into and advanced through the needle assembly from the proximal end outside the patient. Advancement of the stylet 106 will cause the catheter to emerge from the needle assembly 100 out through the slot therein until the stylet has been inserted all the way to the distal end of the needle assembly and the first catheter has been deflected entirely outside of the assembly 100.

Subsequently, the needle assembly 100 is readjusted back to the first (closed) configuration, to once more define the window 154. Then a second catheter can be inserted from the second skin location S2, and advanced through the tunneller into the window 154, and from there further into the target tissue (e.g. the dura mater) as already described. When such a second catheter is to be inserted, rather than completely withdraw the tunneller after insertion of the first catheter it can be simply modestly withdrawn so that its distal end no longer enters the window 154 of the needle assembly 100, where it might interfere with actuating the same. Once the first catheter has been placed and the needle assembly re-adjusted back to its first configuration, the tunneller can be re-advanced so that its distal end just enters the window 154 in order to redirect the second catheter to be introduced therein.

By using the needle assembly 100 in conjunction with the conventional tunneller 200, the location where the catheter(s) 300 enter(s) the skin (i.e., the second skin location ‘S2’) is laterally offset and remote from the puncture location ‘M’ in the spinal dura mater, thus minimizing the potential for a migratory infection to reach the dura mater puncture site. Moreover, the depth at which the catheter 300 is tunneled underneath the skin surface can be adjusted based on the angle at which the tunneller is inserted, and deeper tunneled conduits can be accommodated using the disclosed techniques. This is because it no longer is necessary to access the tunneller sheath distal end via a skin incision, in order to fish the free end of the catheter retrograde therethrough to come out the other end. Instead, the catheter is inserted via that sheath in the first instance, from the remote location where the tunneller is inserted, thus obviating the need to maintain a local skin incision in the vicinity of the dura-mater puncture, and for the tunneled sheath to be shallow enough for easy access through such an incision. The disclosed needle assembly 100 reduces the number of steps required for such a tunneling procedure, and facilitates performing the procedure more efficiently in a manner that does not require snaking the catheter out from a skin wound in the vicinity of the intended puncture site.

While the invention described above has been discussed with respect to inserting a catheter into the spinal dura, the needle assembly 100 could also be used to insert a wire or other flexible-elongated surgical implement or tool to reach and/or treat other target tissues at other locations within the body for other purposes; including both the delivery and withdrawal of fluids, delivery of therapeutic agents, and placement of sensors. Moreover, the scale of the needle assembly 100 also can be adapted to different indications. For example, the needle assembly 100 can be of comparable scale to a conventional Touhy needle when used to place spinal catheters, which is the example indication primarily described herein. But the needle assembly 100 also can be scaled to a larger diameter and/or greater length (or alternatively a smaller diameter and/or smaller length) to accommodate other clinical indications. For example, a larger-diameter needle assembly 100 compared to a conventional Tuohy needle may be used to insert a large-diameter catheter or tube into the urologic, gastrointestinal or respiratory systems to facilitate the introduction or withdrawal of fluids (in the latter case, said fluid potentially being oxygen or air for breathing), as well as other organ systems. In a specific non-limiting example, a larger-diameter assembly 100 compared to a conventional Tuohy needle could be used to insert a suprapubic catheter to accommodate a tunneling procedure as described above.

The invention has been described with reference to example embodiments. Modifications and alterations thereto will be evident to persons of skill in the art upon a reading and understanding this specification.

What is claimed is:

1. An introducer needle assembly comprising:
a hollow outer shaft, and
a hollow inner shaft nested within said outer shaft;
said inner shaft being repositionable relative to said outer shaft between a first orientation and a second orientation when the inner shaft is fully inserted within the outer shaft,
wherein in said first orientation, the inner and outer shafts define a window adjacent to a proximal end of said outer shaft that provides access to a common channel defined within the inner and outer shafts, and
wherein in said second orientation, the inner and outer shafts define a slot that provides access to said common channel, said slot extending from adjacent to said proximal end to a distal end of said outer shaft.

2. The assembly of claim 1, wherein said inner shaft is removable from said outer shaft, and wherein said inner shaft is rotatable between said first and said second orientations.

3. The assembly of claim 1, wherein said outer shaft has an outer cutout extending longitudinally in a circumferential wall thereof, and wherein said inner shaft comprises a circumferential wall having a slotted portion and a window portion, said slotted portion comprising an inner cutout extending longitudinally in the circumferential wall of the inner shaft such that said circumferential wall is discontinuous and has a first arc length in said slotted portion, said circumferential wall being confined to a narrow wall segment having a second circumferential arc length in said window portion, said second circumferential arc length being less than said first circumferential arc length.

4. The assembly of claim 3, wherein said slotted portion is disposed at a distal end of the inner shaft, and the window portion is disposed proximally from said slotted portion.

5. The assembly of claim 3, wherein in said first orientation, the inner and outer cutouts of said inner and outer shafts, respectively, are not aligned, wherein said window is defined in-part by an edge of the circumferential wall in the slotted portion of said inner shaft traversing the outer cutout in said outer shaft.

6. The assembly of claim 3, wherein in said second orientation, the inner and outer cutouts of said inner and outer shafts, respectively, are aligned to thereby define the slot.

7. The assembly of claim 1, further comprising a first head affixed to a proximal end of the outer shaft and a second head affixed to a proximal end of the inner shaft, said first head having a bore and said second head having a neck that is received within said bore.

8. The assembly of claim 7, wherein a boss is formed on an inner circumferential wall of the bore of the first head and projects radially inward, wherein said neck of the second head has a radially recessed detent extending along a circumferential arc segment thereof and defined between opposing first and second stops, and wherein said boss is received within said detent.

9. The assembly of claim 8, wherein in said first orientation the boss engages the first stop, and wherein in said second orientation the boss engages the second stop.

10. The assembly of claim 8, said first head having a boss indicator aligned relative to the boss, and said second head having circumferentially spaced first and second status indicators, wherein in said first orientation, said boss indicator is aligned with said first status indicator, and wherein in said second orientation, the boss indicator is aligned with said second status indicator.

11. The assembly of claim 1, further comprising a solid stylet removably nested within said inner shaft.

12. The assembly of claim 11, wherein the outer shaft and the stylet have respective tips formed at distal ends thereof, wherein the tip of the stylet is formed complimentary to the tip of the outer shaft.

13. A method of introducing a catheter into a desired location of a patient, said method comprising the steps of:
inserting an introducer needle assembly into the patient at a first skin location and along a first trajectory targeting a predetermined target tissue region within a body of the patient, said introducer needle assembly being in a first configuration wherein a window is defined adjacent to a proximal end thereof that provides access to a channel therein;
inserting a tunneller at a second skin location, remote from the first skin location, and advancing the tunneller subcutaneously along a second trajectory that is angled with respect to the first trajectory and targeting the window;
advancing a catheter through the tunneller along the second trajectory and through said window; and
redirecting the catheter from the second trajectory along the first trajectory within said channel, and advancing the catheter from its point of entry at the second skin location, along the second and first trajectories successively, until a distal end of the catheter reaches the target tissue region.

14. The method of claim 13, further comprising, after the distal end of the catheter has reached the target tissue region, adjusting the introducer needle assembly to a second configuration wherein a slot now extends from said proximal end to a distal end thereof; and
at least partially withdrawing the introducer needle assembly from the patient at the first skin location wherein the catheter passes through said slot and remains in place with its distal end in the target tissue region.

15. The method of claim 13, wherein the redirection of the catheter from along said second trajectory to along said first trajectory is achieved entirely subcutaneously.

16. The method of claim 13, where the target tissue region is within the patient's spinal dura mater.

17. The method of claim 14, wherein said adjusting step includes reorienting an inner shaft of the introducer needle assembly with respect to an outer shaft of the introducer needle assembly.

18. The method of claim 13, wherein said tunneller is advanced along the second trajectory such that a distal end thereof is received through said window and within the channel, and wherein the second trajectory forms an angle of or greater than 90° with respect to the first trajectory.

19. An introducer needle assembly comprising:
a hollow outer shaft having an outer cutout extending longitudinally in a circumferential wall thereof from adjacent a proximal end thereof to a distal end thereof;
a first head affixed to the proximal end of the outer shaft, said first head having a bore, wherein a boss is formed on an inner circumferential wall of the bore and projects radially inward;
a hollow inner shaft nested within said outer shaft, said inner shaft comprising a circumferential wall having a slotted portion and a window portion, said slotted portion comprising an inner cutout extending longitudinally in the circumferential wall of the inner shaft such that said circumferential wall is discontinuous and has a first arc length in said slotted portion, said circumferential wall being confined to a narrow wall segment having a second circumferential arc length in said window portion, said second circumferential arc length being less than said first circumferential arc length; and
a second head affixed to a proximal end of the inner shaft, said second head having a neck that is received within said bore, said neck having a radially recessed detent extending along a circumferential arc segment thereof and defined between opposing first and second stops, wherein said boss is received within said detent,
said inner shaft being rotatable relative to said outer shaft between a first orientation and a second orientation when the inner shaft is fully inserted within the outer shaft,
wherein in said first orientation:
the inner and outer shafts define a window adjacent to the proximal end of the outer shaft that provides access to a common channel defined within the inner and outer shafts,
the inner and outer cutouts of said inner and outer shafts, respectively, are not aligned, wherein said window is defined in-part by an edge of the circumferential wall in the slotted portion of said inner shaft traversing the outer cutout in said outer shaft, and
the boss engages the first stop, and
wherein in said second orientation:
the inner and outer shafts define a slot that provides access to said common channel, said slot extending from adjacent to said proximal end of said outer shaft to said distal end of said outer shaft,
the inner and outer cutouts of said inner and outer shafts, respectively, are aligned to thereby define the slot, and
the boss engages the second stop.

20. The assembly of claim 19, further comprising a stylet removably nested within said inner shaft, said stylet being non-hollow.
